(19) United States
(12) Patent Application Publication
(10) Pub. No.: US 2023/0320966 A1
(43) Pub. Date: Oct. 12, 2023

(54) Personal Care Compositions

(71) Applicant: Colgate-Palmolive Company, New York, NY (US)

(72) Inventors: Robert GRUYTCH, Roselle Park, NJ (US); Shyamala PILLAI, Hillsborough, NJ (US)

(73) Assignee: Colgate-Palmolive Company, New York, NY (US)

(21) Appl. No.: 18/042,484

(22) PCT Filed: Aug. 27, 2021
(86) PCT No.: PCT/US2021/047874

§ 371 Date: Feb. 22, 2023

Related U.S. Application Data
(60) Provisional application No. 63/072,391, filed on Aug. 31, 2020.

(51) Int. Cl. A61K 8/58; A61K 8/31; A61K 8/25; A61K 8/04; A61Q 15/00

(52) U.S. Cl. CPC A61K 8/585; A61K 8/31; A61K 8/25; A61K 8/046; A61Q 15/00; A61K 2800/34; A61K 2800/612

(57) ABSTRACT

Described herein are aerosol compositions useful for deodorant and/or antiperspirant applications. Such compositions are substantially free of cyclomethicone. Also described are methods of making and using the same.

### CROSS-REFERENCE TO RELATED APPLICATIONS

The present application claims the benefit of priority to U.S. Provisional Patent Application Ser. No. 63/072,391, filed Aug. 31, 2020, the entirety of which is incorporated herein by reference.

### BACKGROUND

Washing, cleansing and caring for one's own body is a fundamental human need, and modern industry is constantly trying to meet these needs of man in a variety of ways. Particularly important for daily hygiene is the elimination, or at least reduction, of body odor. Body odor arises to a large extent from the bacterial decomposition of individual components of perspiration on the skin.

Numerous specific deodorant and antiperspirant personal care products are known in the art which have been developed for use in body regions having a high density of sweat glands, particularly in the underarm region and on the feet. These products have been formulated in a variety of dosage forms, such as in stick form, pump spray, liquid and gel roll on application, cream, as a soaked flexible substrate (towels), and as aerosol spray.

Aerosols are mixtures of propellant and a base mixture, the base also commonly referred to as the concentrate. The base is a mixture of materials that are often classified as a volatile material. Typical materials used in a base are, for example, a cyclomethicone such as decamethylcyclopentasiloxane (D5), or isododecane. However, use of cyclomethicone has been identified by regulatory agencies as potentially harmful to the environment due to its stability and aversion to breaking down.

There is a need for an aerosol deodorant and/or antiperspirant that is substantially free, or free, of cyclomethicone and which can still yield superior performance, such as providing consumer sensory and efficacy benefits, providing a tight area of spraying, and giving a high deposition of active ingredients.

In some embodiments, the present invention solves these previous technical problems. More particularly, it relates to aerosol compositions useful for deodorant and/or antiperspirant applications where such compositions are substantially free, or free, of cyclomethicone.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The present invention relates to deodorant and/or antiperspirant compositions that can be dispensed by aerosol. Accepting the concept that an aerosol spray product consists of an aerosol concentrate or base, and a propellant or a propellant mixture, and that a given material has a wt % that can be expressed as the wt % in the concentrate or in the can, leads to reporting that wt % in both terms. We will use this concept and report the wt % or wt % range as in-concentrate followed by in parentheses the wt % in-can. And will use the ratio of 15 wt % concentrate in the can to 85 wt % propellant in the can as an example, although this ratio can range from 5 wt % to about 50 wt % concentrate and 50 wt % to about 95 wt % propellant.

Applicants have discovered a personal care composition, comprising: a volatile compound having the formula (CH3)₂CH(CH2)₇₋₉CH₃; an organic derivative of hectorite clay; and a silicon-containing gum blend comprising a dimethicone and a dimethiconol. Such compositions are cyclomethicone-free, or substantially free thereof, and useful for application as an aerosol for deodorant and/or antiperspirant. In certain embodiments, the volatile compound is isododecane. In certain embodiments, the volatile compound is present in an amount of from about 5 wt. % to about 75 wt. % (0.75 wt % to about 11.25 wt %), 5 wt. % to about 60 wt. % (0.75 wt % to about 9.0 wt %), 5 wt. % to about 20 wt. % (0.75 wt % to about 3.0 wt %), or 40 wt. % to about 65 wt. % (6.0 wt % to about 9.75 wt %). In certain embodiments, the personal care composition is according to any foregoing embodiment, wherein the derivative of hectorite clay is selected from stearalkonium hectorite, disteardimonium hectorite, and combinations thereof. In certain embodiments, the personal care composition is according to any foregoing embodiment, wherein the derivative of hectorite clay is present in an amount of from about 1 wt. % to about 5 wt. % (0.15 wt % to about 0.75 wt %), from about 1 wt. % to about 4 wt. % (0.15 wt % to about 0.60 wt %), or from about 2 wt. % to about 5 wt. % (0.30 wt % to about 0.75 wt %). In certain embodiments, the silicon-containing gum blend has a viscosity of about between 6,000 cP to about 16,000 cP at 25° C. In further embodiments, the silicon-containing gum blend is present in an amount of from about 2 wt. % to about 15 wt. % (0.30 wt % to about 2.25 wt %), from about 2 wt. % to about 10 wt. % (0.30 wt % to about 1.50 wt %), from about 2 wt. % to about 8 wt. % (0.30 wt % to about 1.20 wt %), or from about 6 wt. % to about 10 wt. % (0.90 wt % to about 1.50 wt %). In preferred embodiments, the composition is substantially free, or is free, of cyclomethicone. In certain embodiments, the personal care composition according to any foregoing embodiment, wherein the composition further comprises an emollient, and wherein the emollient is optionally selected from isopropyl myristate, isopropyl palmitate, myristyl palmitate, dimethicone, diheptyl succinate and capryloyl glycerin/sebacic acid copolymer, octyldodecanol, PPG-14 Butyl Ether, and C12-15 Alkyl Benzoate and combinations thereof. In certain embodiments, the emollient is present in an amount of from about 2 wt. % to about 40 wt. % (0.30 wt % to about 6.0 wt %), from about 2 wt. % to about 25 wt. % (0.30 wt % to about 3.75 wt %), from about 2 wt. % to about 10 wt. % (0.30 wt % to about 1.50 wt %), from about 2 wt. % to about 8 wt. % (0.30 wt % to about 1.20 wt %), or from about 4 wt. % to about 8 wt. % (0.60 wt % to about 1.20 wt %). In certain embodiments, the personal care composition according to any foregoing embodiment, wherein the dimethicone is present up to 16 wt. % (2.40 wt %). In certain embodiments, the composition is selected from deodorant, antiperspirant, and combinations of two or more thereof. In certain preferred embodiments, the composition is an aerosol.

In further embodiments, the invention is a method of treating, preventing or ameliorating a symptom associated with a disease, disorder or condition of a keratinous substance, comprising applying a personal care composition according to any foregoing embodiment to a keratinous substance of a subject in need thereof. In certain embodiments, the keratinous substance is selected from: skin, hair, and combinations thereof.

In other embodiments, the invention is a personal care composition, comprising: an organic derivative of hectorite clay; and a silicon-containing gum blend comprising a dimethicone and a dimethiconol; wherein the dimethicone is present at up to 16 wt. % of the composition. In certain embodiments, the derivative of hectorite clay is selected from stearalkonium hectorite, disteardimonium hectorite, and combinations thereof. In certain embodiments, the derivative of hectorite clay is present in an amount of from about 1 wt. % to about 5 wt. % (0.15 wt % to about 0.75 wt %), from about 1 wt. % to about 4 wt. % (0.15 wt % to about 0.60 wt %), or from about 2 wt. % to about 5 wt. % (0.30 wt % to about 0.75 wt %). In certain embodiments, the silicon-containing gum blend has a viscosity of about between 6,000 cP to about 16,000 cP at 25° C. In certain embodiments, the personal care composition is according to any one of the embodiments within this paragraph, and wherein the silicon-containing gum blend is present in an amount of from about 2 wt. % to about 15 wt. % (0.30 wt % to about 2.25 wt %), from about 2 wt. % to about 10 wt. % (0.30 wt % to about 1.50 wt %), or from about 2 wt. % to about 8 wt. % (0.30 wt % to about 1.20 wt %). In certain embodiments, the personal care composition is according to any one of the embodiments within this paragraph, and wherein the composition is substantially free, or is free, of cyclomethicone. In certain embodiments, the personal care composition is according to any one of the embodiments within this paragraph, and wherein the composition further comprises an emollient, wherein the emollient is optionally selected from isopropyl myristate, isopropyl palmitate, myristyl palmitate, dimethicone, diheptyl succinate and capryloyl glycerin/sebacic acid copolymer, octyldodecanol, PPG-14 Butyl Ether, and C12-15 Alkyl Benzoate and combinations thereof. In certain embodiments, the emollient is present in an amount of from about 2 wt. % to about 40 wt. % (0.30 wt % to about 6.0 wt %), from about 2 wt. % to about 25 wt. % (0.30 wt % to about 3.75 wt %), from about 2 wt. % to about 10 wt. % (0.30 wt % to about 1.50 wt %), from about 2 wt. % to about 8 wt. % (0.30 wt % to about 1.20 wt %), or from about 4 wt. % to about 8 wt. % (0.60 wt % to about 1.20 wt %). In certain embodiments, the personal care composition is according to any one of the embodiments within this paragraph, and wherein the composition is selected from deodorant, antiperspirant, and combinations of two or more thereof. In certain preferred embodiments, the composition is an aerosol.

In further embodiments, the invention is a method of treating, preventing or ameliorating a symptom associated with a disease, disorder or condition of a keratinous substance, comprising applying a personal care composition according to any one of the embodiments described in the preceding paragraph to a keratinous substance of a subject in need thereof. In further embodiments, the keratinous substance is selected from: skin, hair, and combinations thereof.

In further embodiments, the invention is a personal care composition comprising: a volatile compound having the formula (CH3)₂CH(CH2)₇₋₉CH₃; and a silicon-containing gum blend comprising a dimethicone and a dimethiconol. In certain embodiments, the volatile compound is isododecane. In further embodiments, the volatile compound is present in an amount of from about 5 wt. % to about 75 wt. % (0.75 wt % to about 11.25 wt %), 5 wt. % to about 60 wt. % (0.75 wt % to about 9.0 wt %), 5 wt. % to about 20 wt. % (0.75 wt % to about 3.0 wt %), or 40 wt. % to about 65 wt. % (6.0 wt % to about 9.75 wt %). In certain embodiments, the personal care composition is according to any one of the embodiments within this paragraph, and wherein the silicon-containing gum blend has a viscosity of about between 6,000 cP to about 16,000 cP at 25° C. In certain embodiments, the personal care composition is according to any one of the embodiments within this paragraph, and wherein the silicon-containing gum blend is present in an amount of from about 2 wt. % to about 15 wt. % (0.30 wt % to about 2.25 wt %), from about 2 wt. % to about 10 wt. % (0.30 wt % to about 1.50 wt %), or from about 2 wt. % to about 8 wt. % (0.30 wt % to about 1.20 wt %). In certain embodiments, the personal care composition is according to any one of the embodiments within this paragraph, and wherein the composition is substantially free, or is free, of cyclomethicone. In certain embodiments, the personal care composition is according to any one of the embodiments within this paragraph, and wherein the composition further comprises an emollient, wherein the emollient is optionally selected from isopropyl myristate, isopropyl palmitate, myristyl palmitate, dimethicone, diheptyl succinate and capryloyl glycerin/sebacic acid copolymer, octyldodecanol, PPG-14 Butyl Ether, and C12-15 Alkyl Benzoate and combinations thereof. In further embodiments, the emollient is present in an amount of from about 2 wt. % to about 40 wt. % (0.30 wt % to about 6.0 wt %), from about 2 wt. % to about 25 wt. % (0.30 wt % to about 3.75 wt %), from about 2 wt. % to about 10 wt. % (0.30 wt % to about 1.50 wt %), from about 2 wt. % to about 8 wt. % (0.30 wt % to about 1.20 wt %), or from about 4 wt. % to about 8 wt. % (0.60 wt % to about 1.20 wt %). In certain embodiments, the personal care composition is according to any one of the embodiments within this paragraph, and wherein the dimethicone is present up to 16 wt. % (2.40 wt %). In certain embodiments, the personal care composition is according to any one of the embodiments within this paragraph, and wherein the composition is selected from deodorant, antiperspirant, and combinations of two or more thereof. In certain preferred embodiments, the composition is an aerosol.

In further embodiments, the invention is a method of treating, preventing or ameliorating a symptom associated with a disease, disorder or condition of a keratinous substance, comprising applying a personal care composition according to any one of the embodiments of the preceding paragraph to a keratinous substance of a subject in need thereof. In certain embodiments, the keratinous substance is selected from: skin, hair, and combinations thereof.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

According to the present application, the term “about” means +/−5% of the reference value. As used herein, the term “substantially free” is intended to mean an amount less than about 1 wt. %; preferably less than about 0.5 wt. %, and more preferably less than about 0.25 wt. % of the composition. As used herein, the term “free” is intended to mean that no detectable amount exists within the composition.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

An “antiperspirant” is a composition that mitigates body odor as well as prevents sweating by affecting sweat glands. An antiperspirant may be applied to any part of the body and is generally applied to the underarms. In preferred embodiments, the antiperspirant is applied to the person's underarm or feet. The term “antiperspirant composition” refers to any composition containing an antiperspirant active and which is intended to be applied onto skin.

The term “antiperspirant efficacy” refers to the amount of wetness protection provided by application of an antiperspirant composition to a keratinous substance on a subject, such as the underarm area (or axilla).

A “deodorant” is a composition that is applied to the body of a person to prevent body odor caused by the bacterial breakdown of perspiration. A deodorant may be applied to any part of the body. Under selected embodiments, the deodorant is applicable to a person's underarm and feet.

The term “propellant” refers to one or more gases that are used to pressurize the antiperspirant composition to facilitate egress of the antiperspirant composition from the container. Some propellants may be a mixture of gases (e.g., a mixture of isobutane, butane and propane). A propellant may be in the form of a liquid (i.e., a liquefied gas) when under pressure within the reservoir of a spray device. In addition, a propellant may be in its gaseous state within the head space of the reservoir. A propellant may be present in both a liquefied form and its gaseous state within the reservoir. Unless specified otherwise (e.g., liquid propellant or gaseous propellant), the term propellant is intended to encompass the liquefied form and the gaseous state individually and collectively.

The term “volatile” refers to a material that has a boiling point less than 250° C. (at atmospheric pressure) and/or a vapor pressure of about 0.1 mm Hg at 25° C.

Personal care compositions of the invention are useful as aerosols for deodorant and/or antiperspirant application.

In certain embodiments, the personal care compositions are anhydrous. The anhydrous composition is free, or substantially free, of water. In preferred embodiments, the composition contains water in an amount of less than 10.0 wt %, less than 5.0 wt %, less than 3.0 wt %, less than 1.0 wt %, less than 0.1 wt %, less than 0.05 wt %, less than 0.01 wt %, less than 0.005 wt %, or less than 0.0001 wt %, based on a total weight of the composition. In certain preferred embodiments, the composition has less than 1% by weight of free water, less than 0.1% by weight of free water, has no free water, or has no added water apart from any water bound to the active antiperspirant ingredient.

The compositions of the present invention control perspiration and optionally, also body odor, from localized areas of the body, such as the underarm. In one embodiment, the present invention is directed to a personal care composition, comprising: a volatile compound having the formula (CH₃)₂CH(CH₂)₇₋₉CH₃; a derivative of hectorite clay; and a silicon-containing gum blend comprising a dimethicone and a dimethiconol. In certain embodiments, the volatile compound is isododecane. In certain embodiments, the volatile compound is present in an amount of from about 15 wt. % to about 75 wt. % (2.25 wt % to about 11.25 wt %), from about 15 wt. % to about 60 wt. % (2.25 wt % to about 9.0 wt %), or from about 15 wt. % to about 55 wt. % (2.25 wt % to about 8.25 wt %).

In certain embodiments, the derivative of hectorite clay are modified clays such as hectorites modified by C₁₀₋₂₂ ammonium chloride, hectorite modified by distearyl dimethyl ammonium chloride such as marketed, for example, under the trade name Bentone® 27 or Bentone® 38 (Elementis plc). In certain embodiments, the derivative of hectorite clay is selected from stearalkonium hectorite, disteardimonium hectorite, and a combination thereof. In certain embodiments, the derivative of hectorite clay is present in an amount of from about 2 wt. % to about 20 wt. % (0.3 wt. % to about 3.0 wt %), 2 wt. % to about 10 wt. % (0.3 wt % to about 1.5 wt %), 2 wt. % to about 5 wt. % (0.3 wt % to about 0.75 wt %), 3 wt. % to about 10 wt. % (0.45 wt % to about 1.5 wt %), from about 3 wt. % to about 8 wt. % (0.45 wt % to about 1.2 wt %), or from about 3 wt. % to about 6 wt. % (0.45 wt % to about 0.9 wt %).

The personal care compositions of the present invention comprise a gum blend. Gum Blend materials are used because they are often heavier in weight than the volatile materials and therefore may enhance the delivery of the personal care composition to the necessary area. For example, gum blend materials may provide adhesion of the base mixture to the skin. Gum blend materials may include dimethiconol, a viscous material dispersed in for example, cyclomethicone or dimethicone. In certain embodiments, the silicon-containing gum blend has a viscosity of about between 6,000 cP to about 16,000 cP at 25° C. In certain embodiments, the silicon-containing gum blend is present in an amount of from about 2 wt. % to about 15 wt. % (0.3 wt % to about 2.25 wt %), from about 2 wt. % to about 10 wt. % (0.3 wt % to about 1.5 wt %), from about 2 wt. % to about 8 wt. % (0.3 wt % to about 1.2 wt %), from about 4 wt. % to about 10 wt. % (0.6 wt % to about 1.5 wt %), or from about 4 wt. % to about 8 wt. % (0.6 wt % to about 1.2 wt %).

In certain embodiments, the composition is substantially free of cyclomethicone. In certain embodiments, the composition is free of cyclomethicone.

In certain embodiments, the composition further comprises an emollient. In certain embodiments, the emollient may comprise alkyl or alkyl-aryl ester oils having a boiling point of above 150° C. and a melting point below 20° C. Such ester oils include oils containing one or two alkyl groups of 12 to 24 carbon atoms length, including isopropyl myristate, isopropyl palmitate and myristyl palmitate. Other ester oils include alkyl or aryl benzoates such C₁₂₋₁₅ alkyl benzoate, for example Finsolv® TN or Finsolv® Sun. In certain embodiments, the emollient is selected from isopropyl myristate, isopropyl palmitate, myristyl palmitate, dimethicone, Finsolv® TN, Finsolv® Sun, diheptyl succinate and capryloyl glycerin/sebacic acid copolymer (sold as LexFeel™ N50, Inolex, Inc.), octyldodecanol, PPG-14 Butyl Ether or combinations thereof. In certain embodiments, the emollient is present in an amount of from about 2 wt. % to about 40 wt. % (0.3 wt % to about 6.0 wt %), from about 2 wt. % to about 30 wt. % (0.3 wt % to about 4.5 wt %), from about 2 wt. % to about 25 wt. % (0.3 wt % to about 3.75 wt %), from about 2 wt. % to about 10 wt. % (0.3 wt % to about 1.5 wt %), from about 2 wt. % to about 8 wt. % (0.3 wt % to about 1.2 wt %), from about 4 wt. % to about 8 wt. % (0.6 wt % to about 1.2 wt %), from about 6 wt. % to about 20 wt. % (0.9 wt % to about 3.0 wt %), or from about 6 wt. % to about 15 wt. % (0.9 wt % to about 2.25 wt %). In certain embodiments, the emollient is isopropyl palmitate and is present in an amount of from about 2 wt. % to about 40 wt. % (0.3 wt % to about 6.0 wt %), from about 2 wt. % to about 30 wt. % (0.3 wt % to about 4.5 wt %), from about 2 wt. % to about 25 wt. % (0.3 wt % to about 3.75 wt %), from about 2 wt. % to about 10 wt. % (0.3 wt % to about 1.5 wt %), from about 2 wt. % to about 8 wt. % (0.3 wt % to about 1.2 wt %), from about 4 wt. % to about 8 wt. % (0.6 wt % to about 1.2 wt %), from about 5 wt. % to about 20 wt. % (0.75 wt % to about 3.0 wt %), or from about 6 wt. % to about 15 wt. % (0.9 wt % to about 2.25 wt %).

In some embodiments, the personal care composition further comprises gelling agents and/or emollients. Examples of other gelling agents/emollients include, but are not limited to, waxes, esters of fatty acid and fatty alcohol, triglycerides, partially or fully hydrogenated soybean oil, partially or fully hydrogenated castor oil, other partial or fully hydrogenated plant oils, stearyl alcohol, propylene carbonate, or other cosmetically acceptable materials, which are solid or semi-solid at room temperature and provide a consistency suitable for application to the skin.

In certain embodiments, the composition is selected from deodorant, antiperspirant, and a combination thereof. In certain embodiments, the composition is an aerosol.

The personal care composition of the present invention utilizes one or more active antiperspirant ingredients. In some embodiments, the at least one active antiperspirant ingredient is selected from aluminum salts, zirconium salts and zinc salts. Any of the known aluminum containing antiperspirant active materials can be utilized in the composition of the invention. Antiperspirant actives include, but are not limited to, aluminum chlorohydrate, aluminum chloride, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum, sesquichlorohydrate polyethylene glycol, aluminum sesquichlorohydrate propylene glycol, aluminum-zirconium octachlorohydrate, aluminum-zirconium octachlorohydrex gly, aluminum-zirconium pentachlorohydrate, aluminum-zirconium pentachlorohydrex gly, aluminum-zirconium tetrachlorohydrate, aluminum-zirconium tetrachlorohydrex gly, aluminum-zirconium trichlorohydrate, aluminum-zirconium trichlorohydrex gly, and combinations thereof. In some embodiments, the active ingredient is aluminium chlorohydrate, aluminum sesquichlorohydrate, aluminum zirconium tetrachlorohydrex GLY, and mixtures thereof. In certain embodiments, the antiperspirant ingredient is preferably present in the composition of the invention in an amount of from about 2% to about 60% by weight (0.6 wt % to about 9.0 wt %, from about 8% to about 55% by weight (1.2 wt % to about 8.25 wt %), from about 10% to about 50% by weight (1.5 wt % to about 7.5 wt %), from about 20% to about 50% by weight (3.0 wt % to about 7.5 wt %), or from about 30% to about 50% by weight (4.5 wt % to about 7.5 wt %), based on the total weight of the composition. Antiperspirant actives can be incorporated into compositions in amounts of 0.5 to 25 w % (on an actives basis) of the final composition, but the amount used will depend on the formulation of the composition. In certain embodiments, the active ingredient is present in the composition of the invention in an amount of from about 2% to about 10% by weight, from about 5% to about 8% by weight, or from about 5% to about 7% by weight, based on the total weight of the composition.

In certain embodiments, the composition may include any known deodorant active. Examples of deodorant actives include, but are not limited to, antimicrobial actives, alcohols, 2,4,4′-trichloro-T-hydroxy diphenyl ether (Triclosan), benzethonium chloride, polyhexamethylene biguanides, triethylcitrate, 2-amino-2-methyl-1-propanol (AMP), cetyl-trimethylammomium bromide, cetyl pyridinium chloride, farnesol (3,7,11-trimethyl-2,6,10-dodecatrien-1-ol), N-(4-chlorophenyl)-N′-(3,4-dichlorophenyl)urea (Triclocarban), silver halides, octoxyglycerin (Sensiva™ SC 50) and various zinc salts (for example, zinc ricinoleate), bactericides, and/or bacteriostats. The deodorant active can, illustratively, be included in the composition in an amount of 0-5% wt %, or 0.01-1 wt %, based on the total weight of the composition including the propellant.

Additional components of the antiperspirant compositions optionally include any components suitable for use in such compositions which are known in the art. Optionally, the composition further comprises one or more ingredients selected from: a fragrance, a preservative, an emulsifier, an antioxidant and an emollient.

In certain embodiments, the personal care compositions comprise a preservative. Examples of suitable preservatives include benzyl alcohol, piroctone olamine, phenoxyethanol, parabens, pentanediol, benzoic acid/sodium benzoate, sorbic acid/potassium sorbate, and other organic acids used to provide antimicrobial protection. Preservation boosting ingredients include anisic acid, lactic acid, sorbitan caprylate, ethylhexylglycerin, caprylyl glycol, octanediol, and similar substances. Suitable preservatives are the substances listed in the International Cosmetic Ingredient Dictionary and Handbook, 9th Edition with the function “preservatives”. In at least one embodiment, the preservative is selected from the group consisting of phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin and combinations thereof. In at least one embodiment, the composition comprises a preservative selected from the group consisting of cetyltrimethyl ammoniumchloride, cetylpyridinium chloride, benzethonium chloride, diisobutylethoxyethyldimethyl benzylammoniumchloride, sodium N-lauryl sarcosinate, sodium-N-palmethylsarcosinate, lauroylsarcosine, N-myristoylglycine, potassium-N-laurylsarcosine, trimethylammoniumchloride, sodium aluminum chlorohydroxylactate, triethylcitrate, tricetylmethylammoniumchloride, 2,4,4′-trichloro-2′-hydroxydiphenylether (Triclosan), phenoxyethanol, 1,5-pentandiol, 1,6-hexandiol, 3,4,4′-trichlorocarbanilide (Triclocarban), diaminoalkylamide, L-lysine hexadecylamide, heavy metal citrate salts, salicylate, piroctose, zinc salts, pyrithione and its heavy metal salts, zinc pyrithione, zinc phenol sulfate, farnesol, ketoconazol, oxiconazol, bifonazole, butoconazole, cloconazole, clotrimazole, econazole, enilconazole, fenticonazole, isoconazole, miconazole, sulconazole, tioconazole, fluconazole, itraconazole, terconazole, naftifine, terbinafine, selenium disulfide, Octopirox®, methylchloroisothiazolinone, methylisothiazolinone, methyldibromo glutaronitrile, AgCl, chloroxylenol, sodium salts of diethylhexylsulfosuccinate, sodiumbenzoate, phenoxyethanol, benzylalkohol, phenoxyisopropanol, paraben, such as butyl-, ethyl-, methyl- and propylparaben, and their salts, pentandiol, 1,2-octanediol, ethylhexylglycerin, benzylalcohol, sorbic acid, benzoic acid, lactic acid, imidazolidinyl urea, diazolidinyl urea, dimethylol dimethyl hydantoin (DMDMH), sodium salts of hydroxymethyl glycinate, hydroxyethylglycine of sorbic acid and combinations thereof. In at least one embodiment, the preservative is selected from the group consisting of phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin and combinations thereof. In certain embodiments, the preservative is present in an amount of from about 0.1 wt. % to about 5 wt. % (0.015 wt % to about 0.75 wt %), from about 0.5 wt. % to about 3 wt. % (0.075 wt % to about 0.45 wt %), from about 0.7 wt. % to about 1.5 wt. % (0.105 wt % to about 0.225 wt %), or from about 0.1 wt. % to about 1.5 wt. % (0.015 wt % to about 0.225 wt %).

In certain embodiments, the personal care composition further comprises a polysaccharide. In certain embodiments, the polysaccharide comprises fructan, inulin, xanthan gum, guar gum, alginates, xyloses, carboxymethylcellulose, and combinations thereof. In certain embodiments, the polysaccharide is present in an amount of from about 0.01 wt. % to about 5 wt. % (about 0.0015 wt % to about 0.75 wt %), from about 0.05 wt. % to about 3 wt. % (0.0075 wt % to about 0.45 wt %), from about 0.05 wt. % to about 1 wt. % (about 0.0075 wt % to about 0.15 wt %), or from about 0.05 wt. % to about 0.8 wt. % (about 0.0075 wt % to about 0.12 wt %).

In certain embodiments, the personal care composition further comprises a humectant. In certain embodiments, the humectant is selected from: glycerin; butyloctanol; hyaluronic acid; urea; sodium lactate; propylene glycol; glycolic acid; sorbitol; and a combination of two or more thereof. In further embodiments, the humectant is selected from glycerin; butyloctanol; and a combination thereof. In some embodiments, the humectant is present in an amount of from about 0.05 wt. % to about 10 wt. % (about 0.0075 wt % to about 1.5 wt %), from about 1 wt. % to about 8 wt. % (about 0.15 wt % to about 1.2 wt %), or from about 5 wt. % to about 8 wt. % (about 0.75 wt % to about 1.2 wt %). In some embodiments, the humectant is butyloctanol. In certain embodiments, the humectant is 2-butyloctanol. In certain embodiments, the butyloctanol may be present in an amount ranging from about 0.01 wt. % to about 8 wt. % (about 0.0015 wt % to about 1.2 wt %) based on the total weight of the personal care composition. In some embodiments, the butyloctanol may be present in an amount ranging from about 1 wt. % to about 8 wt. % (about 0.15 wt % to about 1.2 wt %) based on the total weight of the personal care composition. In some embodiments, the butyloctanol may be present in an amount ranging from about 5 wt. % to about 8 wt. % (about 0.75 wt % to about 1.2 wt %) based on the total weight of the personal care composition. In some embodiments, the butyloctanol may be present in an amount up to 8 wt. % based on the total weight of the personal care composition. In some embodiments, the butyloctanol may be present in an amount up to 7 wt. % based on the total weight of the personal care composition.

The aerosol composition is packaged in a dispenser comprising an aerosol container capable of dispensing the composition topically on to the axillary area of a subject. Such containers are known in the art. Typically, the container is a pressurized aerosol container. The composition may further comprise at least one propellant which is a liquefied gas, typically a liquefied hydrocarbon such as one or more of butane, propane and isobutane, for example a mixture of these three propellant components. The at least one propellant is typically present in an amount of from about 50 to 95 wt %, for example about 85 wt %, based on the total weight of the composition.

The droplet particle size distribution of the aerosol may be measured using a Malvern SprayTec instrument (Malvern Analytical, Malvern, UK) and using the technique of laser diffraction. In preferred embodiments, the droplet particle size distribution should not be greater than 125 μm, preferably not greater than 100 μm, more preferably not greater than 80 μm. In preferred embodiments, the droplet particle size distribution should not be smaller than 5 μm, more preferably not smaller than 3 μm. This is to ensure the product spray will not have a percentage of droplets too small which may be hazardous to consumer health.

The present invention is also directed to a method of preventing or mitigating body odor and/or wetness comprising applying a personal care composition according to any one of embodiments described herein to the skin or keratinous surface of a subject in need thereof. In certain embodiments of the method, the keratinous surface is selected from: skin, hair, and a combination of two or more thereof.

In some embodiments, the present invention is also directed to a method of treating, preventing or ameliorating a symptom associated with a disease, disorder or condition of a keratinous substance, comprising applying a personal care composition according to any one of the embodiments described herein to a keratinous substance of a subject in need thereof. In some embodiments, the disease, disorder or condition of a keratinous substance is selected from: body odor or sweating. In some embodiments, the keratinous substance is selected from: skin, hair, and a combination thereof.

In some embodiments, the personal care compositions described herein further comprise flaxseed oil. In other embodiments, the personal care compositions described herein further comprise a natural oil selected from: almond oil; olive oil, and the like. In some embodiments, the personal care compositions described herein comprise aloe leaf juice.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

Embodiment compositions and performance. Compositions comprising isododecane, isopropyl palmitate, and dimethicones in dimethiconol were evaluated for aerosol performance. Briefly, to determine the area of spray, the aerosol was sprayed onto a 10″×10″ piece of plexiglass from a distance of 6″ for 3 seconds. The diameter of the spray outline was measured in centimeters horizontally and vertically across the plexiglass. The average of the two diameters was then applied to the formula πr² to determine the area of the circle as the spray produced a deposit in a circular shape. This is an important parameter because a tighter, smaller spray area means more of the spray is targeted to the underarm, providing a more efficacious product.

To determine the amount deposited, steps similar to the spray pattern protocol were performed with the exception that a piece of black cloth was taped to the plexiglass sheet. The cloth was weighed before and after the can contents were sprayed onto it. Likewise, the can was weighed before and after the spray event. The time of spray was also measured. Taking the weight difference of the can will indicate how much was sprayed from the can. Taking the weight difference of the cloth will tell how much of the spray landed on the target area. Dividing the weight difference of product deposited on the cloth by the weight difference dispensed by the can and multiplying by 100 gives the % product deposited. This is an important parameter as it indicates how much of the concentrate, the portion of the can product that contains active for sweat and malodor protection, is targeted to the underarm area and provides an indication of sample efficacy.

Viscosity was measured at 25° C. The results are shown in Table 1. Ideally one wants to maximize the amount of product deposited while minimizing the Area of Spray, while balancing that against a viscosity level that will be able to be filled into cans. When analyzing these results statistically versus the aforementioned constraints one finds a small window of favorable material levels that provides the desired outcomes.

TABLE 1

| | | | | Average | Average | |
|---|---|---|---|---|---|---|
| | | | | Area of | % Product | Vis- |
| Com- | Iso- | Isopropyl | Belsil | Spray | Concentrate | cosity |
| position | dodecane | Palmitate | 3096 | (cm2) | Deposited | (cps) |
| Trial 1 | 31.417 | 4.167 | 6.917 | 86.98 | 61.62 | 24,000 |
| Trial 2 | 30.333 | 6.333 | 5.833 | 126.58 | 71.56 | 31,000 |
| Trial 3 | 32.667 | 5.417 | 4.417 | 95.95 | 62.67 | 38,000 |
| Trial 4 | 30.333 | 6.333 | 5.833 | 96.69 | 63.72 | 69,000 |
| Trial 5 | 32.667 | 4.167 | 5.667 | 82.65 | 71.81 | 22,500 |
| Trial 6 | 27.667 | 8.167 | 6.667 | 109.06 | 63.36 | 68,000 |
| Trial 7 | 27.667 | 7.917 | 6.917 | 121.29 | 64.46 | 42,000 |
| Trial 8 | 32.500 | 2.000 | 8.000 | 138.41 | 64.10 | 18,000 |
| Trial 9 | 29.917 | 8.167 | 4.417 | 81.22 | 60.96 | 68,000 |
| Trial 10 | 25.000 | 9.500 | 8.000 | 110.25 | 62.52 | 38,000 |
| Trial 11 | 29.500 | 10.000 | 3.000 | 96.62 | 74.91 | 98,000 |
| Trial 12 | 35.000 | 2.000 | 5.500 | 152.12 | 61.85 | 33,000 |
| Trial 13 | 25.000 | 10.000 | 7.500 | 96.55 | 65.57 | 82,000 |
| Trial 14 | 35.000 | 4.500 | 3.000 | 95.46 | 54.40 | 29,000 |

### Example 2

Compositions comprising a) isododecane (IDD); and one or more of b) isopropyl palmitate (IPP), c) dimethicone, d) dimethiconol in dimethicone (Belsil® 3096, viscosity of 6000 mPa s at 25° C., or Belsil® 1020, viscosity of 1600 mPa s at 25° C., Wacker), e) C₁₁₋₁₃ isoparaffin in dimethiconol, isohexadecane, and dimethicone (PMX-1502, Dow), and f) dimethicone and dimethiconol (PMX-1503, viscosity of 1395 mPa s at 25° C., Dow) were evaluated for aerosol performance. Samples were analyzed as discussed above. The results are shown in Table 2 (below).

TABLE 2

| | Ave. | | Ave. Spray |
|---|---|---|---|
| | Area of | Concentrate | Droplet Particle |
| Composition | Spray (cm²) | Deposited | Size (μm) |
| IDD - IPP | 122.95 | 43.16 | 25.44 |
| IDD - IPP - Belsil ® | 99.43 | 56.38 | 26.71 |
| 3096 | | | |
| IDD - IPP - PMX- | 79.93 | 56.64 | 33.78 |
| 1502 | | | |
| IDD - IPP - PMX- | 102.51 | 49.39 | 30.78 |
| 1503 | | | |
| IDD - IPP - Belsil ® | 117.89 | 50.34 | 31.57 |
| 1020 | | | |
| IDD - Dimethicone - | 83.91 | 63.4 | 26.52 |
| PMX-1502 | | | |
| IDD - Dimethicone - | 88.5 | 55.21 | 19.36 |
| Belsil ® 3096 | | | |

Use of IDD and IPP alone provided for a large area of spray in combination with a low concentrate of deposit. Surprisingly, compositions having the addition of dimethiconol in dimethicone improved both the area of spray and the concentrate deposited. Further, the higher viscosity dimethiconol in dimethicone solution provided a tighter area of spray and high deposition. Use of C₁₁₋₁₃ isoparaffin in dimethiconol, isohexadecane, and dimethicone provided for particularly tight area of spray and strong deposition.

### Example 3

Table 3 describes suitable concentration ranges for exemplary embodiments of personal care compositions of the present invention. It has been discovered that exemplary compositions of the present invention, encompassed by the formulations reported in Table 3, provide unexpected improvements in the average area of spray (cm²), concentrate deposited, and average spray droplet particle size (μm) without the need for a cyclomethicone.

TABLE 3

| Ingredient | Wt. % |
|---|---|
| Volatile compound | 15-60 |
| Emollient | 5-10 |
| Gum Blend | 5-10 |
| Active Material | 4-60 |
| Hectorite clay derivative | 1-25 |

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. A personal care composition, comprising:
a volatile compound having the formula (CH3)₂CH(CH2)₇₋₉CH₃;
an organic derivative of hectorite clay; and
a silicon-containing gum blend comprising:
a dimethicone; and
a dimethiconol.

2. The personal care composition according to claim 1, wherein the volatile compound is isododecane.

3. The personal care composition according to claim 1, wherein the volatile compound is present in an amount of from about 5 wt. % to about 75 wt. %, 5 wt. % to about 60 wt. %, 5 wt. % to about 20 wt. %, or 40 wt. % to about 65 wt. % in the aerosol concentrate, or about 0.75 wt % to about 11.25 wt %, about 0.75 wt % to about 9.0 wt %, about 0.75 wt % to about 3.0 wt % or about 6.0 wt % to about 9.75 wt %, when calculated in the presence of a propellant (ratio of 15% wt % concentrate and 85 wt % propellant).

4. The personal care composition according to claim 1, wherein the derivative of hectorite clay is selected from stearalkonium hectorite, disteardimonium hectorite, and combinations thereof.

5. The personal care composition according to claim 1, wherein the derivative of hectorite clay is present in an amount of from about 1 wt. % to about 5 wt. %, from about 1 wt. % to about 4 wt. %, or from about 2 wt. % to about 5 wt. % in the aerosol concentrate, or about 0.15 wt % to about 0.75 wt %, about 0.15 wt % to about 0.6 wt %, or about 0.3 wt % to about 0.75 wt %, when calculated in the presence of a propellant (ratio of 15% wt % concentrate and 85 wt % propellant).

6. The personal care composition according to claim 1, wherein the silicon-containing gum blend has a viscosity of about between 6,000 cP to about 16,000 cP at 25° C.

7. The personal care composition according to claim 1, wherein the silicon-containing gum blend is present in an amount of from about 2 wt. % to about 15 wt. %, from about 2 wt. % to about 10 wt. %, from about 2 wt. % to about 8 wt. %, or from about 6 wt. % to about 10 wt. % in the aerosol concentrate, or about 0.3 wt % to about 2.25 wt %, about 0.3 wt % to about 1.5 wt %, about 0.3 wt % to about 1.2 wt %, or about 0.9 wt % to about 1.5 wt %, when calculated in the presence of a propellant (ratio of 15% wt % concentrate and 85 wt % propellant).

8. The personal care composition according to claim 1, wherein the composition is substantially free, or is free, of cyclomethicone.

9. The personal care composition according to claim 1, wherein the composition further comprises an emollient, wherein the emollient is optionally selected from isopropyl myristate, isopropyl palmitate, myristyl palmitate, dimethicone, diheptyl succinate and capryloyl glycerin/sebacic acid copolymer, and combinations thereof.

10. The personal care composition according to claim 9, wherein the emollient is present in an amount of from about 2 wt. % to about 40 wt. %, from about 2 wt. % to about 25 wt. %, from about 2 wt. % to about 10 wt. %, from about 2 wt. % to about 8 wt. %, or from about 4 wt. % to about 8 wt. % in the aerosol concentrate, or about 0.3 wt % to about 6.0 wt %, about 0.3 wt % to about 3.75 wt %, about 0.3 wt % to about 1.5 wt %, about 0.3 wt % to about 1.2 wt %, or about 0.6 wt % to about 1.2 wt %, when calculated in the presence of a propellant (ratio of 15% wt % concentrate and 85 wt % propellant).

11. (canceled)

12. (canceled)

13. (canceled)

14. (canceled)

15. (canceled)

16. A personal care composition, comprising:
an organic derivative of hectorite clay; and
a silicon-containing gum blend comprising:
a dimethicone; and
a dimethiconol;
wherein the dimethicone is present at up to 16 wt. % of the composition.

17. The personal care composition according to claim 16, wherein the derivative of hectorite clay is selected from stearalkonium hectorite, disteardimonium hectorite, and combinations thereof.

18. The personal care composition according to claim 16, wherein the derivative of hectorite clay is present in an amount of from about 1 wt. % to about 5 wt. %, from about 1 wt. % to about 4 wt. %, or from about 2 wt. % to about 5 wt. % in the aerosol concentrate, or about 0.15 wt % to about 0.75 wt %, about 0.15 wt % to about 0.6 wt %, or about 0.3 wt % to about 0.75 wt %, when calculated in the presence of a propellant (ratio of 15% wt % concentrate and 85 wt % propellant).

19. The personal care composition according to claim 16, wherein the silicon-containing gum blend has a viscosity of about between 6,000 cP to about 16,000 cP at 25° C.

20. The personal care composition according to claim 16, wherein the silicon-containing gum blend is present in an amount of from about 2 wt. % to about 15 wt. %, from about 2 wt. % to about 10 wt. %, or from about 2 wt. % to about 8 wt. % in the aerosol concentrate, or about 0.3 wt % to about 2.25 wt %, about 0.3 wt % to about 1.5 wt %, or about 0.3 wt % to about 1.2 wt %, when calculated in the presence of a propellant (ratio of 15% wt % concentrate and 85 wt % propellant).

21. (canceled)

22. (canceled)

23. (canceled)

24. (canceled)

25. (canceled)

26. (canceled)

27. (canceled)

28. A personal care composition, comprising:
a volatile compound having the formula (CH3)₂CH(CH2)₇₋₉CH₃; and
a silicon-containing gum blend comprising:
a dimethicone; and
a dimethiconol.

29. The personal care composition according to claim 28, wherein the volatile compound is isododecane.

30. The personal care composition according to claim 28, wherein the volatile compound is present in an amount of from about 5 wt. % to about 75 wt. %, 5 wt. % to about 60 wt. %, 5 wt. % to about 20 wt. %, or 40 wt. % to about 65 wt. % in the aerosol concentrate, or about 0.75 wt % to about 11.25 wt %, about 0.75 wt % to about 9.0 wt %, about 0.75 wt % to about 3.0 wt %, or about 6.0 wt % to about 9.75 wt %, when calculated in the presence of a propellant (ratio of 15% wt % concentrate and 85 wt % propellant).

31. The personal care composition according to claim 28, wherein the silicon-containing gum blend has a viscosity of about between 6,000 cP to about 16,000 cP at 25° C.

32. The personal care composition according to claim 28, wherein the silicon-containing gum blend is present in an amount of from about 2 wt. % to about 15 wt. %, from about 2 wt. % to about 10 wt. %, or from about 2 wt. % to about 8 wt. % in the aerosol concentrate, or about 0.3 wt % to about 2.25 wt %, about 0.3 wt % to about 1.5 wt %, or about 0.3 wt % to about 1.2 wt %, when calculated in the presence of a propellant (ratio of 15% wt % concentrate and 85 wt % propellant).

33. (canceled)

34. (canceled)

35. (canceled)

36. (canceled)

37. (canceled)

38. (canceled)

39. (canceled)

40. (canceled)
